(19) United States
(12) Patent Application Publication
(10) Pub. No.: US 2003/0191051 A1
(43) Pub. Date: Oct. 9, 2003

(54) Combination therapy for the treatment of bacterial infections

(76) Inventors: Philip Needleman, Creve Coeur, MO (US); Barry Hafkin, Danbury, CT (US)

Correspondence Address: PHARMACIA & UPJOHN, 301 HENRIETTA ST, 0228-32-LAW, KALAMAZOO, MI, 49007, US

(21) Appl. No.: 10/348,300

(22) Filed: Jan. 21, 2003

Related U.S. Application Data
(60) Provisional application No. 60/351,058, filed on Jan. 23, 2002.

(51) Int. Cl. A61K 38/14; A61K 31/704; A61K 31/496; A61K 31/416; A61K 31/4709; A61K 31/545; A61K 31/43

(52) U.S. Cl. 514/8; 514/37; 514/192; 514/200; 514/253.08; 514/312; 514/300; 514/406

(57) ABSTRACT

The present invention provides compositions and methods for treating or preventing bacterial infections. The compositions and methods include the use of antibiotics and cyclooxygenase inhibitors.

### CROSS-REFERENCE TO RELATED APPLICATION

This application claims the benefit of the following provisional application(s): U.S. Serial No. 60/351,058, filed January 23, 2002, under 35 USC 119(e)(i).

### BACKGROUND OF THE INVENTION

Antibiotics were introduced into medical practice nearly 50 years ago. Antibiotics have been used to control many life-threatening diseases, to reduce death and illness, and to increase the life expectancy of the population. However, the benefits of antibiotics have not been gained without the introduction of some associated problems.

Antibiotics are commonly administered to treat bacterial infections by, for example, injection, oral administration, or application to the skin in ointment form. Many antibiotics are potent anti-infective agents, but also cause toxic side effects. For example, penicillin is highly allergenic and can cause skin rashes, shock, and other allergic responses. Tetracyclines are capable of causing major changes in the intestinal bacterial population and can result in superinfection by fungi and other microorganisms. Chloramphenicol is known to produce severe blood diseases, which has led to restrictions in its use. Streptomycin can result in ear and kidney damage. Moreover, many antibiotics have lost their effectiveness against some bacterial diseases and, as a result, some illnesses that were once easily treatable now pose treatment problems for physicians and their patients.

Because of these problems with known antibiotics, the medical community is continually searching for and developing new approaches for treating bacterial infections. Such approaches include, for example, the development of new classes of antibiotics and improved methods of administering known antibiotics. Specifically, there is a need in the medical arts for methods to treat bacterial infections in mammals by administering sufficient amounts of potent antibiotics while minimizing undesirable side effects.

### SUMMARY OF THE INVENTION

In one embodiment, the present invention provides a method of treating or preventing a bacterial infection in a mammal. The method includes administering to the mammal (a) a pharmaceutically effective amount of an antibiotic or a pharmaceutically acceptable salt thereof; and (b) a pharmaceutically effective amount of a cyclooxygenase inhibitor or a pharmaceutically acceptable salt or derivative or prodrug thereof. Preferably, the cyclooxygenase inhibitor is a cyclooxygenase-2 selective inhibitor. Preferably, the mammal is a human or an animal, more preferably a human. Preferably, the antibiotic or pharmaceutically acceptable salt thereof, and the cyclooxygenase inhibitor or pharmaceutically acceptable salt or derivative or prodrug thereof, are administered at least once per day. Preferably, the antibiotic is linezolid. Preferably, the cyclooxygenase inhibitor is celecoxib or rofecoxib.

In another embodiment, the present invention provides a method for reducing side effects of an antibiotic in a mammal. The method includes administering to a mammal a sufficient amount of an antibiotic or a pharmaceutically acceptable salt thereof to result in side effects; and administering to the mammal a pharmaceutically effective amount of a cyclooxygenase-2 selective inhibitor or a pharmaceutically acceptable salt or derivative or prodrug thereof to reduce the side effects. Preferably, the antibiotic is linezolid and the cyclooxygenase inhibitor is celecoxib or rofecoxib.

In another embodiment, the present invention provides a composition including an antibiotic or a pharmaceutically acceptable salt thereof; and an effective amount of a cyclooxygenase-2 selective inhibitor or a pharmaceutically acceptable salt or derivative or prodrug thereof. Preferably, the antibiotic is linezolid and the cyclooxygenase inhibitor is celecoxib or rofecoxib.

In another embodiment, the present invention provides a kit including a container; an antibiotic or a pharmaceutically acceptable salt thereof in the container; and an effective amount of a cyclooxygenase-2 selective inhibitor or a pharmaceutically acceptable salt or derivative or prodrug thereof in the container. Preferably, the antibiotic is linezolid and the cyclooxygenase inhibitor is celecoxib or rofecoxib.

The present invention provides advantages over known methods of treating bacterial infections with antibiotics. For example, acceptable dosages of some antibiotics are practically limited by the severity of undesirable side effects. Treatment of a bacterial infection with (a) a pharmaceutically effective amount of an antibiotic or a pharmaceutically acceptable salt thereof; and (b) a pharmaceutically effective amount of a cyclooxygenase inhibitor or a pharmaceutically acceptable salt or derivative or prodrug thereof may result in reduced side effects as compared to the antibiotic administered alone. Alternatively, treatment of a bacterial infection with (a) a pharmaceutically effective amount of an antibiotic or a pharmaceutically acceptable salt thereof; and (b) a pharmaceutically effective amount of a cyclooxygenase inhibitor or a pharmaceutically acceptable salt or derivative or prodrug thereof may allow for administration of higher dosages of the antibiotic without resulting in increased side effects. While not wishing to be bound by theory, it is believed that when antibiotic treatment of a mammal causes endotoxins to be released, the release sets off a tumor necrosis factor-alpha (TNF-A) mediated response that can be blocked by the cyclooxygenase inhibitor.

Definitions

The term “antibiotic” refers to an antibacterial agent. A “pharmaceutically effective” amount of an antibiotic is an amount sufficient to provide the intended treatment in the body being treated (e.g., to treat or prevent a bacterial infection in a mammal). A pharmaceutically effective amount of an antibiotic may also result in undesirable side effects including, for example, itching, swelling, inflammation, and death.

The term “gram-positive antibiotic” refers to an antibacterial agent active against gram-positive bacterial organisms.

The term “gram-negative antibiotic” refers to an antibacterial agent active against gram-negative bacterial organisms.

The terms “cyclooxygenase inhibitor” or “COX inhibitor” interchangeably refer to a therapeutic compound with inhibits the enzyme cyclooxygenase. Cyclooxygenase inhibitors include, for example, cyclooxygenase-inhibiting non-steroidal anti-inflammatory drugs (NSAIDs) and cyclooxygenase-2 selective inhibitors. A “pharmaceutically effective” amount of a cyclooxygenase inhibitor is an amount sufficient to provide the intended treatment in the body being treated (e.g., to treat or prevent inflammation in a mammal).

The terms “cyclooxygenase-2 selective inhibitor” and “COX-2 selective inhibitor” interchangeably refer to a therapeutic compound that selectively inhibits the COX-2 isoform of the enzyme cyclooxygenase. In practice, COX-2 selectivity varies depending on the conditions under which the test is performed and on the inhibitors being tested. However, for the purposes of this patent, COX-2 selectivity can be measured as a ratio of the in vitro or in vivo IC₅₀ value for inhibition of COX-1, divided by the IC₅₀ value for inhibition of COX-2. A COX-2 selective inhibitor is any inhibitor for which the ratio of COX-1 IC₅₀ to COX-2 IC₅₀ is greater than about 1, preferably at least about 5, more preferably at least about 10, still more preferably at least about 50, and more preferably still at least about 100.

Compounds disclosed in the present application may be used in their native forms or as salts. In cases where forming a stable nontoxic acid or base salt is desired, administration of the compound as a pharmaceutically acceptable salt may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, etoglutarate, and glycerophosphate. Suitable inorganic salts may also be formed, including hydrochloride, hydrobromide, sulfate, nitrate, bicarbonate, and carbonate salts.

Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598.

The following definitions are used, unless otherwise described: halo is fluoro, chloro, bromo, or iodo.

The term “alkoxy” refers to —O-alkyl groups. Alkyl, alkoxy, etc. denote both straight and branched groups; but reference to an individual radical such as “propyl” embraces only the straight chain radical, a branched chain isomer such as “isopropyl” being specifically referred to. Unless otherwise specifically stated alkyl moieties include between 1 and 6 carbon atoms. When alkyl can be partially unsaturated, the alkyl chain may include one or more (e.g. 1, 2, 3, or 4) double or triple bonds in the chain.

The term “alkenyl” refers to both straight- and branched-chain moieties containing at least one —C═C—. Unless otherwise specifically stated alkenyl moieties include between 1 and 6 carbon atoms.

The term “alkynyl” refers to both straight- and branched-chain moieties containing at least one —C≡C—. Unless otherwise specifically stated alkynyl moieties include between 1 and 6 carbon atoms, between 1 and 6 carbon atoms

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkyl moieties will include between 3 and 9 carbon atoms and at least one —C═C— group within the cyclic ring.

The term “amino” refers to —NH₂.

The term “aryl” denotes a phenyl radical or an ortho-fused bicyclic carbocyclic radical having about nine to ten ring atoms in which at least one ring is phenyl.

The term “het” is a five- (5), six- (6), or seven- (7) membered saturated or unsaturated ring containing 1, 2, 3, or 4 heteroatoms selected from the group consisting of non-peroxide oxygen, sulfur, and nitrogen; as well as a radical of an ortho-fused bicyclic heterocycle of about eight to twelve ring atoms derived therefrom, particularly a benz-derivative or one derived by fusing a propylene, trimethylene, tetramethylene or another monocyclic het diradical thereto. Het also includes “heteroaryl,” which encompasses a radical attached via a ring carbon of a monocyclic aromatic ring containing five or six ring atoms consisting of carbon and 1, 2, 3, or 4 heteroatoms each selected from the group consisting of non-peroxide oxygen, sulfur, and N(X) wherein X is absent or is H, O, C₁₋₄alkyl, phenyl or benzyl. The term “het” may be an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds disclosed in the present application having a chiral center may exist in, and be isolated in, optically active and racemic forms. Some compounds may exhibit polymorphism. Compounds disclosed in the present application encompass any racemic, optically-active, polymorphic, tautomeric, or stereoisomeric form, or mixture thereof, of the compound that possesses the useful properties described herein. It is well known in the art how to prepare optically active forms (e.g., by resolution of the racemic form through recrystallization techniques, by synthesis from optically-active starting materials, by chiral synthesis, or by chromatographic separation using a chiral stationary phase) and how to determine antibacterial activity using standard tests or other tests that are well known in the art.

The carbon atom content of various hydrocarbon-containing moieties is indicated by a prefix designating a lower and upper number of carbon atoms in the moiety, i.e., the prefix Cᵢ₋ⱼ indicates a moiety of the integer “i” to the integer “j” carbon atoms, inclusive. Thus, for example, C₁₋₇alkyl refers to alkyl of one to seven carbon atoms, inclusive.

Compounds disclosed in the present application are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “rt” for room temperature).

Specific and preferred values listed below for radicals, substituents, and ranges, are for illustration only; they do not exclude other defined values or other values within defined ranges for the radicals and substituents. Compounds disclosed in the present application include compounds having any combination of values, specific values, more specific values, and preferred values described herein.

More specifically, alkyl can be methyl, ethyl, propyl, isopropyl, butyl, iso-butyl, sec-butyl, pentyl, 3-pentyl, hexyl, or heptyl; C₃₋₈cycloalkyl can be cyclopropyl, cyclobutyl, cyclopentyl, cyclohexyl, cycloheptyl, or cyclooctyl; C₁₋₇alkoxy can be methoxy, ethoxy, propoxy, isopropoxy, butoxy, iso-butoxy, sec-butoxy, pentoxy, 3-pentoxy, hexyloxy, 1-methylhexyloxy, or heptyloxy; C(═O)C₁₋₇alkyl can be acetyl, propanoyl, butanoyl, pentanoyl, 4-methylpentanoyl, hexanoyl, or heptanoyl.

Specificall, aryl includes, but are not limited to, phenyl, indenyl, or naphthyl.

Specifically, het includes, but are not limited to, pyridinyl, piperidinyl, morpholino, thiomorpholino, furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide); more sepficically, het includes pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

When alkyl is partially unsaturated, it can specifically be vinyl, allyl, 1-propenyl, 2-propenyl, 1-butenyl, 2-butenyl, 3-butenyl, 1,3-butadienyl, 1-pentenyl, 2-pentenyl, 3-pentenyl, 4-pentenyl, 1-hexenyl, 2-hexenyl, 3-hexenyl, 4-hexenyl, 5-hexenyl, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, or 5-hexynyl.

### DETAILED DESCRIPTION OF THE INVENTION

The present application discloses a combination therapy that includes the treatment of a subject with (a) an antibiotic or a pharmaceutically acceptable salt thereof; and (b) a pharmaceutically effective amount of a cyclooxygenase inhibitor or a pharmaceutically acceptable salt or derivative or prodrug thereof. The combination preferably results in the effective treatment of, for example, a bacterial infection relative to previously disclosed treatment regimens.

For combination therapy, an antibiotic or a pharmaceutically acceptable salt thereof may be administered concurrently or concomitantly with a cyclooxygenase inhibitor or a pharmaceutically acceptable salt or derivative or prodrug thereof. The term “concurrently” means the subject being treated takes one drug within about 5 minutes of taking the other drug. The term “concomitantly” means the subject being treated takes one drug within the same treatment period of taking the other drug. The same treatment period is preferably within about 48 hours, more preferably within about twelve hours.

For the combination therapy, an antibiotic or a pharmaceutically acceptable salt thereof, and a cyclooxygenase inhibitor or a pharmaceutically acceptable salt or derivative or prodrug thereof may be administered in the same physical form or separately, i.e., they may be administered in the same delivery vehicle or in different delivery vehicles.

Antibiotics

Gram-positive Antibiotics. In combating infective diseases caused by gram-positive organisms, gram-positive antibiotics may be used alone or in combination with other antibiotics that are active against gram-positive organisms. Some gram-positive antibiotics may also have activity against gram-negative organisms. Representative examples of gram-positive antibiotics are listed in Table 1.

TABLE 1
Gram-Positive Antibiotics For Use In Combination Therapy

| AGENTS | LO DOSE | HI DOSE | STD DOSE |
|---|---|---|---|
| OXAZOLIDINONES | | | |
| Linezolid | 2 mg | 600 mg | 200-400 mg |
| | | | (oral) |
| Linezolid | | | 2-4 mg (IV) |
| AMINOGLYCOSIDES | | | |
| Amikacin | | | 15 mg/kg/day |
| Gentamicin | 1 mg/kg/day | 5 mg/kg/day | |
| | .5 mg/kg | 2.5 mg/kg | |
| Spectinomycin | | | 40 mg/kg |
| Tobramycin | 1 mg/kg/day | 5 mg/kg/day | |
| | .5 mg/kg/day | 5 mg/kg/day | |
| PENEMS | | | |
| Imipenem/cilastatin | 62.5 mg | 1 g | |
| | 6.25 mg/kg | 25 mg/kg | |
| Meropenem | | | 40 mg/kg |
| | .5 mg/kg | 2.5 mg/kg | |
| 1^{st} GEN CEPHS | | | |
| Cefadroxil | .25 g/day | 2 g/day | |
| | | | 30 mg/kg/day |
| Cefazolin | 62.5 mg | 1.5 g | |
| | 6.25 mg/kg/day | 100 mg/kg/day | |
| Cephalexin | 62.5 mg | 500 mg | |
| | 6.25 mg/kg/day | 50 mg/kg/day | |
| 2^{ND} GEN CEPHS | | | |
| Cefaclor | 62.5 mg | 500 mg | |
| | 5 mg/kg/day | 40 mg/kg/day | |
| Cefotetan | 0.125 g | 3 g | |
| | 10 mg/kg/day | 80 mg/kg/day | |
| Cefoxitin | .25 g | 3 g | |
| | 20 mg/kg/day | 160 mg/kg/day | |
| Cefprozil | 62.5 mg | 500 mg | |
| | 1.87 mg/kg/dose | 15 mg/kg/dose | |
| Cefuroxime | 187.5 mg | 3 g | |
| | 31.25 mg | 500 mg | |
| | 12.5 mg/kg/day | 150 mg/kg/day | |
| | 31.25 mg/kg/day | 500 mg/kg/day | |
| Loracarbef | 50 mg | 400 mg | |
| | 3.75 mg/kg/day | 500 mg/kg/day | |
| 3^{RD} GEN CEPHS | | | |
| Cefdinir | 75 mg | | 600 mg |
| Cefixime | 50 mg | | 400 mg |
| Cefoperazone | .5 g/day | 12 g/day | |
| | 25 mg/kg/day | 150 mg/kg/day | |
| Cefotaxime | .25 g | 2 g | |
| | 12.5 mg/kg/dose | 300 mg/kg/day | |
| Cefpodoxime | 25 mg | 400 mg | 10 mg/kg/day |
| Ceftazidime | 62.5 mg | 2 g q8 | |
| | 25 mg/kg/day | 150 mg/kg/day | |
| Ceftibuten | 12.25 mg/kg | 400 mg | 400 mg |
| Ceftozoxime | .25 g | 4 g | |
| | 12.5 mg/kg/day | 200 mg/kg/day | |
| Ceftriaxone | 31.25 mg | 2 g | |
| | 12.5 mg/kg/day | 100 mg/kg/day | |
| 4^{TH} GEN CEPHS | | | |
| Cefepime | 0.125 g | 2 g | |
| | 12.5 mg/kg | 50 mg/kg q8 | |
| MACROLIDES | | | |
| Azithromycin | 62.5 mg | 500 mg | |
| | 62.5 mg | 500 mg | |
| Clarithromycin | 62.5 mg | 500 mg | 7.5 |
| | | | mg/kg/day |
| Dirithromycin | | | 500 mg |
| 1^{ST} GEN PENS | | | |
| Penicillin G | 2 million | 30 million units/day | |
| | units/day | | |
| | 2000 units/kg/dy | 400,000 | |
| | | units/kg/day | |
| 2^{ND} GEN PENS | | | |
| Cloxacillin | 62.5 mg | 500 mg | |
| | 12.5 mg/kg/day | 100 mg/kg/day | |
| Dicloxacillin | 31.25 mg | 500 mg | |
| | 3.125 mg/kg/day | 100 mg/kg/day | |
| Nafcillin | 125 mg | 2 g | |
| | 2.5 mg/kg | 25 mg/kg | |
| Oxacillin | 62.5 mg | 2 g | |
| | 125 mg | 1000 mg | |
| | 25 mg/kg/day | 200 mg/kg/day | |
| | 12.5 mg/kg/day | 100 mg/kg/day | |
| 3^{RD} GEN PENS | | | |
| Amoxicillin | 62.5 mg | 875 mg | |
| | 5 mg/kg/day | 45 mg/kg | |
| Amoxicillin/clavulanic | 62.5 mg | 875 mg | |
| acid | | | |
| | 6.25 mg/kg/day | 45 mg/kg/day | |
| Ampicillin | 62.5 mg | 12 g/day q4 | |
| | 6.25 mg/kg/day | 300 mg/kg/day | |
| Ampicillin/sulbactam | 0.375 g | 3 g | 300 |
| | | | mg/kg/day |
| 4^{TH} GEN PENS | | | |
| Mezlocillin | 0.375 g | 4 g | 75 mg/kg |
| Piperacillin | 1.5 g/day | 24 g day | |
| | 25 mg/kg/day | 300 mg/kg/day | |
| Piperacillin/tazobactam | | | 240 |
| | | | mg/kg/day |
| Ticarcillin | .25 g | 4 g | |
| | 12.5 mg/kg/day | 300 mg/kg/day | |
| Ticarcillin/clavulanate | 50 mg/kg/day | 300 mg/kg/day | |
| | 0.775 g | 3.1 g | |
| 1^{ST} GEN QUINOLONES | | | |
| Nalidixic Acid | | | 55 mg/kg/day |
| 2^{ND} GEN | | | |
| QUINOLONES | | | |
| Ciprofloxacin | 50 mg | 750 mg | |
| | 2.5 mg/kg/dose | 15 mg/kg/dose | |
| | 62.5 mg | 750 mg | |
| | 2.5 mg/kg/dose | 15 mg/kg/dose | |
| Enoxacin | 50 mg | 400 mg | |
| Lomefloxacin | | | 400 mg |
| Norfloxacin | | | 400 mg |
| Ofloxacin | 50 mg | 400 mg | |
| 3^{RD} GEN | | | |
| QUINOLONES | | | |
| Levofloxacin | 62.5 mg | 750 mg | |
| Sparfloxacin | 50 mg | 400 mg | |
| 4^{TH} GEN | | | |
| QUINOLONES | | | |
| Alatrofloxacin | 50 mg | 300 mg | |
| Gatifloxacin | 50 mg | 400 mg | |
| Moxifloxacin | | | 400 mg |
| SULFAS | | | |
| Trimethoprim/ | 15 mg | 800 mg | |
| sulfamethoxazole | | | |
| | 3.75 mg/day | 150 mg/day | |
| Sulfisoxazole | 18.75 mg | 150 mg | |
| Sulfamethoxazole | .25 g | 2 g | |
| TETRACYCLINES | | | |
| Doxycycline | 5 mg | 100 mg | |
| Minocycline | 25 mg | 200 mg | |
| Tetracycline | 62.5 mg | 500 mg | |
| OTHER | | | |
| Chloramphenicol | 12.5 mg/kg/day | 100 mg/kg/day | |
| Clindamycin | 150 mg | 900 mg | |
| | 37.5 mg | 450 mg | |
| | 5 mg/kg/day | 40 mg/kg/day | |
| | 2 mg/kg/day | 25 mg/kg/day | |
| Quinupristin/dalfopristin | 1.875 mg/kg | 7.5 mg/kg q8 | |
| Fosfomycin | 3 g | | |
| Nitrofurantoin | 12.5 mg | 100 mg | |
| | 1.25 mg/kg/day | 7 mg/kg/day | |
| Rifampin | 2.5 mg/kg | 600 mg/kg | |
| | 2.5 mg/kg | 600 mg/kg | |
| Trimethoprim | 25 mg | 200 mg | 10 mg/kg/day |
| Vancomycin | | | 1 g |
| | 2.5 mg/kg q6 | 15 mg/kg q8 | |

A particularly preferred gram-positive antibiotic is linezolid:

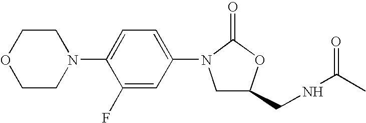

which is commercially available by physicians' prescriptions; and may be made according to U.S. Pat. No. 5,688,792.

Gram-Negative Antibiotics. In combating infective diseases caused by gram-negative organisms, gram-negative antibiotics may be used alone or in combination with other antibiotics that are active against gram-negative organisms. Some gram-negative antibiotics may also have activity against gram-positive organisms. Representative examples of gram-negative antibiotics are listed in Table 2.

TABLE 2
Gram-Negative Antibiotics For Use In Combination Therapy

| AGENTS | LO DOSE | | HI DOSE | | STD DOSE | |
|---|---|---|---|---|---|---|
| AMINOGLYCOSIDES | | | | | | |
| Amikacin | | | | | 15 | mg/kg/day |
| Gentamicin | 0.75 | mg/kg/day | 5 | mg/kg/day | | |
| | 0.5 | mg/kg | 2.5 | mg/kg | | |
| Spectinomycin | | | | | 40 | mg/kg |
| Tobramycin | 0.75 | mg/kg/day | 5 | mg/kg/day | | |
| | 0.5 | mg/kg/day | 5 | mg/kg/day | | |
| PENEMS | | | | | | |
| Imipenem/cilastatin | 62.5 | mg | 1 | g | | |
| | 6.25 | mg/kg | 25 | mg/kg | | |
| Meropenem | | | | | 40 | mg/kg |
| | 0.5 | mg/kg | 2.5 | mg/kg | | |
| 2^{ND} GEN CEPHS | | | | | | |
| Cefaclor | 62.5 | mg | 500 | mg | | |
| | 5 | mg/kg/day | 40 | mg/kg/day | | |
| Cefotetan | 0.125 | g | 3 | g | | |
| | 10 | mg/kg/day | 80 | mg/kg/day | | |
| Cefoxitin | 0.25 | g | 3 | g | | |
| | 20 | mg/kg/day | 160 | mg/kg/day | | |
| Cefprozil | 62.5 | mg | 500 | mg | | |
| | 1.875 | mg/kg/dose | 15 | mg/kg/dose | | |
| Cefuroxime | 187.5 | mg | 3 | g | | |
| | 31.25 | mg | 500 | mg | | |
| | 12.5 | mg/kg/day | 150 | mg/kg/day | | |
| | 31.25 | mg/kg/day | 500 | mg/kg/day | | |
| Loracarbef | 50 | mg | 400 | mg | | |
| | 3.75 | mg/kg/day | 500 | mg/kg/day | | |
| 3^{RD} GEN CEPHS | | | | | | |
| Cefdinir | 75 | mg | | | 600 | mg qd |
| Cefixime | 50 | mg | | | 400 | mg |
| Cefoperazone | 0.25 | g/day | 12 | g/day | | |
| | 25 | mg/kg/day | 150 | mg/kg/day | | |
| Cefotaxime | 0.25 | g | 2 | g | | |
| | 12.5 | mg/kg/dose | 300 | mg/kg/day | | |
| Cefpodoxime | 25 | mg | 400 | mg | 10 | mg/kg/day |
| Ceftazidime | 62.5 | mg | 2 | g q8 | | |
| | 25 | mg/kg/day | 150 | mg/kg/day | | |
| Ceftibuten | 2.25 | mg/kg | 400 | mg | 400 | mg |
| Ceftozoxime | 0.25 | g | 4 | g | | |
| | 12.5 | mg/kg/day | 200 | mg/kg/day | | |
| Ceftriaxone | 31.25 | mg | 2 | g | | |
| | 12.5 | mg/kg/day | 100 | mg/kg/day | | |
| 4^{TH} GEN CEPHS | | | | | | |
| Cefepime | 0.125 | g | 2 | g | | |
| | 12.5 | mg/kg | 50 | mg/kg q8 | | |
| MACROLIDES | | | | | | |
| Azithromycin | 62.5 | mg | 500 | mg | | |
| | 62.5 | mg | 500 | mg | | |
| Clarithromycin | 62.5 | mg | 500 | mg | 7.5 | mg/kg/day |
| Dirithromycin | | | | | 500 | mg |
| 3^{RD} GEN PENS | | | | | | |
| Amoxicillin | 62.5 | mg | 875 | mg | | |
| | 5 | mg/kg/day | 45 | mg/kg | | |
| Amoxicillin/clavulanic acid | 62.5 | mg | 875 | mg | | |
| | 6.25 | mg/kg/day | 45 | mg/kg/day | | |
| Ampicillin | 62.5 | mg | 12 | g/day q4 | | |
| | 6.25 | mg/kg/day | 300 | mg/kg/day | | |
| Ampicillin/sulbactam | 0.375 | g | 3 | g | 300 | mg/kg/day |
| 4^{TH} GEN PENS | | | | | | |
| Mezlocillin | 0.375 | g | 4 | g | 75 | mg/kg |
| Piperacillin | 1.5 | g/day | 24 | g day | | |
| | 25 | mg/kg/day | 300 | mg/kg/day | | |
| Piperacillin/tazobactam | | | | | 240 | mg/kg/day |
| Ticarcillin | 0.25 | g | 4 | g | | |
| | 12.5 | mg/kg/day | 300 | mg/kg/day | | |
| Ticarcillin/clavulanate | 50 | mg/kg/day | 300 | mg/kg/day | | |
| | 0.775 | g | 3.1 | g | | |
| 1^{ST} GEN QUINOLONES | | | | | | |
| Nalidixic Acid | | | | | 55 | mg/kg/day |
| 2^{ND} GEN QUINOLONES | | | | | | |
| Ciprofloxacin | 50 | mg | 750 | mg | | |
| | 2.5 | mg/kg/dose | 15 | mg/kg/dose | | |
| | 62.5 | mg | 750 | mg | | |
| | 2.5 | mg/kg/dose | 15 | mg/kg/dose | | |
| Enoxacin | 50 | mg | 400 | mg | | |
| Lomefloxacin | | | | | 400 | mg |
| Norfloxacin | | | | | 400 | mg |
| Ofloxacin | 50 | mg | 400 | mg | | |
| 3^{RD} GEN QUINOLONES | | | | | | |
| Levofloxacin | 62.5 | mg | 750 | mg | | |
| Sparfloxacin | 50 | mg | 400 | mg | | |
| 4^{TH} GEN QUINOLONES | | | | | | |
| Alatrofloxacin | 50 | mg | 300 | mg | | |
| Gatifloxacin | 50 | mg | 400 | mg | | |
| Moxifloxacin | | | | | 400 | mg |
| SULFAS | | | | | | |
| Trimerhoprim/ | 15/200 | mg | | | | |
| sulfamethoxazole | | | | | | |
| | 3.75 | mg/day | 150 | mg/day | | |
| Sulfisoxazole | 18.75 | mg | 150 | mg | | |
| Sulfamethoxazole | 0.25 | g | 2 | g | | |
| TETRACYCLINES | | | | | | |
| Doxycycline | 5 | mg | 100 | mg | | |
| Minocycline | 25 | mg | 200 | mg | | |
| Tetracycline | 62.5 | mg | 500 | mg | | |
| OTHER | | | | | | |
| Chloramphenicol | 12.5 | mg/kg/day | 100 | mg/kg/day | | |
| Aztreonam | 125 | mg | 2 | g | | |
| | 37.5 | mg | 450 | mg | | |
| | 5 | mg/kg/day | 40 | mg/kg/day | | |
| | 2 | mg/kg/day | 25 | mg/kg/day | | |
| Fosfomycin | | | | | 3 | g |
| Nitrofurantoin | 12.5 | mg | 100 | mg | | |
| | 1.25 | mg/kg/day | 7 | mg/kg/day | | |
| | 2.5 | mg/kg | 600 | mg/kg | | |
| Trimethoprim | 25 | mg | 200 | mg | 10 | mg/kg/day |

All of the above antibiotics are known. They can be either obtained commercially or be prepared according to the references cited in PHYSICIANS' DESK REFERENCE, the 53^{rd} Edition (1999) or the U.S. Food and Drug Administration (FDA) Orange book.

In Tables 1 and 2, the term “Lo Dose” means the recommended lower dosage for the combination therapy of the invention. It may be adjusted even lower depending on the requirements of each subject being treated and the severity of the bacterial infection. The term “Hi Dose” means the recommended highest dosage in the combination therapy. It may be changed hereafter according to the U.S. FDA standard. The term “Std Dose” means the recommended standard dosage for the combination therapy of the present invention. It may be adjusted even lower depending on the requirements of each subject being treated and the severity of the bacterial infection. A specific antibiotic may have more than one recommended dosage range.

Some of the antibiotics disclosed in the present application may further be used with a β-Lactamase inhibitor. For example, imipenem may be used with cilastatin, ampicillin may be used with sulbactam, piperacillin may be used with tazobactam, and ampicillin may be used with sulbactam.

Generally, an antibacterially effective amount of dosage of an antibiotic disclosed in the present application, either administered individually or in combination with other antibiotics, will be in the range of about 0.1 mg/kg of body weight/day to about 400 mg/kg of body weight/day, more preferably about 1.0 mg/kg of body weight/day to about 50 mg/kg of body weight/day. It is to be understood that the dosages of active component(s) may vary depending upon the requirements of each subject being treated and the severity of the bacterial infection.

The desired dose may conveniently be presented in a single dose or as divided into multiple doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

The present invention specifically includes the oxazolidinone antibacterial compounds, which are a novel synthetic class of antimicrobials with potent activity against a number of human and veterinary pathogens.

In some embodiments, antibacterial oxazolidinone compounds have the following formula I:

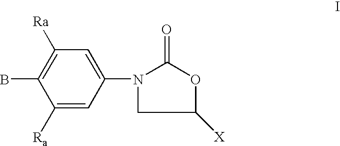

or a pharmaceutically acceptable salt thereof wherein:

B is selected from cycloalkyl, substituted cycloalkyl, cycloalkenyl, substituted cycloalkenyl, aryl, substituted aryl, het and substituted het, or

B and one Rₐ together, with the phenyl carbon atoms to which B and the one Rₐ are bonded, form a het, the het optionally being a substituted het;

X is a group selected from —CH₂—NH—C(O)—R_{b}, —CH₂—NH—C(S)—R_{b}, —CH₂—R_{b}, —CH₂—Y—R_{b};

Each Y is O, S, or —NH—;

Each Rₐ is independently selected from H, alkyl, alkoxy, amino, NO₂, CN, halo, substituted alkyl, substituted alkoxy, and substituted amino; and

Each R_{b} is independently selected from H, —OH, amino, alkyl, substituted alkyl, alkoxy, substituted alkoxy, alkenyl, substituted alkenyl, cycloalkyl, substituted cycloalkyl, cycloalkenyl, substituted cycloalkenyl, het, substituted het, aryl, and substituted aryl.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, and —SNQ₁₀Q₁₀. Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted het” refers to a het moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

Each Q₁₀ is independently selected from —H, alkyl, cycloalkyl, het, cycloalkenyl, and aryl. The het, cycloalkyl, cycloalkenyl, and aryl being optionally substituted with 1-3 substituents selected from halo and Q₁₃.

Each Q₁₁ is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, ═O, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(O)C(Q₁₆)₂OC(O)Q₁₀, —CN, ═O, ═S, —NQ₁₁C(O)Q₁₁, —NQ₁₁C(O)NQ₁₁Q₁₁, —S(O)₂NQ₁₁Q₁₁, —NQ₁₁S(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, cycloalkenyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —CN, —NQ₁₆C(O)Q₁₆, —NQ₁₆C(O)NQ₁₆Q₁₆, —S(O)₂NQ₁₆Q₁₆, and —NQ₁₆S(O)₂Q₁₆. The alkyl, cycloalkyl, and cycloalkenyl being further optionally substituted with ═O or ═S.

Each Q₁₅ is alkyl, cycloalkyl, cycloalkenyl, het, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(O)C(Q₁₆)₂OC(O)Q₁₆, —CN, —NQ₁₆C(O)Q₁₆, —NQ₁₆C(O)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S.

Each Q₁₆ is independently selected from —H, alkyl, and cycloalkyl. The alkyl and cycloalkyl optionally including 1-3 halos.

Other examples of oxazolidinone compounds and methods for producing oxazolidinone compounds may be found, for example, in the following publications which are hereby incorporated by reference in their entirety.

U.S. Pat. Nos. 5,225,565; 5,182,403; 5,164,510; 5,247,090; 5,231,188; 5,565,571; 5,547,950; 5,952,324; 5,968,962; 5,688,792; 6,069,160; 6,239,152; 5,792,765; 4,705,799; 5,043,443; 5,652,238; 5,827,857; 5,529,998; 5,684,023; 5,627,181; 5,698,574; 6,166,056; 6,051,716; 6,043,266; 6,313,307; and 5,523,403.

PCT Application and publications PCT/US93/04850, WO94/01110; PCT/US94/08904, WO95/07271; PCT/US95/02972, WO95/25106; PCT/US95/10992, WO96/13502; PCT/US96/05202, WO96/35691; PCT/US96/12766; PCT/US96/13726; PCT/US96/14135; PCT/US96/17120; PCT/US96/19149; PCT/US97/01970; PCT/US95/12751, WO96/15130, PCT/US96/00718, WO96/23788, WO98/54161, WO99/29688, WO97/30995, WO97/09328, WO95/07271, WO00/21960, WO01/40236, WO99/64417, and WO01/81350.

In certain embodiments, the oxazolidinone can have the formula

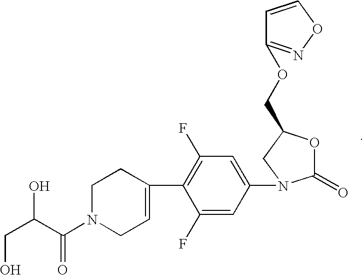

Cyclooxygenase Inhibitors

One of the embodiments of the present invention is a combination therapy including a therapeutic amount of an antibiotic and a therapeutic amount of a cyclooxygenase-inhibiting non-steroidal anti-inflammatory drug (NSAID). Examples of cyclooxygenase-inhibiting NSAIDs include the well-known compounds aspirin, indomethacin, sulindac, etodolac, mefenamic acid, tolmetin, ketorolac, diclofenac, ibuprofen, naproxen, fenoprofen, ketoprofen, oxaprozin, flurbiprofen, nitroflurbiprofen, piroxicam, tenoxicam, phenylbutazone, apazone, or nimesulide or a pharmaceutically acceptable salt or derivative or prodrug thereof. In a preferred embodiment of the invention, the NSAID is selected from the group including indomethacin, ibuprofen, naproxen, flurbiprofen or nitroflurbiprofen. In a still more preferred embodiment of the invention, the NSAID is nitroflurbiprofen.

Cyclooxygenase-2 Selective Inhibitors. Preferably the cyclooxygenase inhibitor is a COX-2 selective inhibitor. In one embodiment of the invention, the COX-2 selective inhibitor is meloxicam, Formula A-1 (CAS registry number 71125-38-7) or a pharmaceutically acceptable salt or derivative or prodrug thereof.

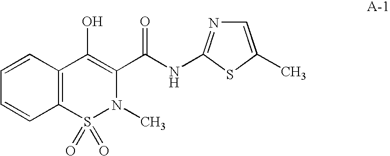

In another embodiment of the invention, the cyclooxygenase-2 selective inhibitor is the COX-2 selective inhibitor RS-57067, 6-[[5-(4-chlorobenzoyl)-1,4-dimethyl-1H-pyrrol-2-yl]methyl]-3(2H)-pyridazinone, Formula A-2 (CAS registry number 179382-91-3) or a pharmaceutically acceptable salt or derivative or prodrug thereof.

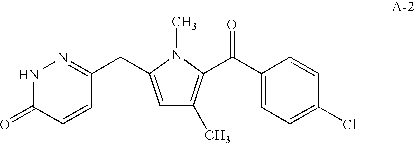

In another embodiment of the invention, the cyclooxygenase-2 selective inhibitor is the COX-2 selective inhibitor ABT-963, 2-(3,4-difluorophenyl)-4-(3-hydroxy-3-methylbutoxy)-5-[4-(methylsulfonyl)phenyl]-(9Cl)-3(2H)-pyridazinone, Formula A-3 (CAS registry number 266320-83-6 or a pharmaceutically acceptable salt or derivative or prodrug thereof.

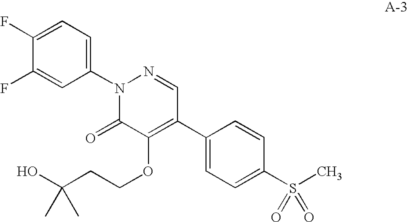

In another embodiment of the invention, the cyclooxygenase-2 selective inhibitor is the COX-2 selective inhibitor COX-189, Formula A-4 (CAS registry number 346670-74-4) or a pharmaceutically acceptable salt or derivative or prodrug thereof.

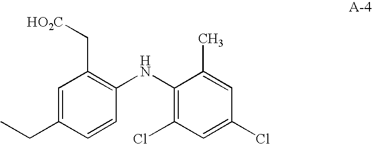

In another embodiment of the invention, the cyclooxygenase-2 selective inhibitor is the COX-2 selective inhibitor NS-398, N-(2-cyclohexyl-4-nitrophenyl)methanesulfonamide, Formula A-5 (CAS registry number 123653-11-2) or a pharmaceutically acceptable salt or derivative or prodrug thereof.

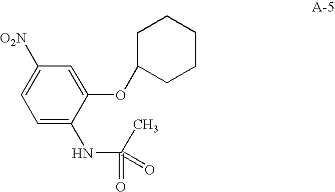

In a preferred embodiment of the invention, the cyclooxygenase-2 selective inhibitor is a COX-2 selective inhibitor of the chromene structural class. For the purposes of the present invention, a chromene class COX-2 selective inhibitor includes substituted benzopyrans, substituted benzothiopyrans, substituted dihydroquinolines, and substituted dihydronaphthyridines having the general Formula:

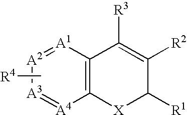

wherein X is selected from O, S, CR^{c}R^{b} and NR^{a};

wherein R^{a} is selected from hydrido, C₁-C₃-alkyl, phenyl-C₁-C₃-alkyl, (substituted phenyl)-C₁-C₃-alkyl, C₁-C₃-alkoxycarbonyl-C₁-C₃-alkyl, and carboxy-C₁-C₆-alkyl;

wherein each of R^{b} and R^{c} is independently selected from hydrido, C₁-C₃-alkyl, substituted or unsubstituted phenyl-C₁-C₃-alkyl, C₁-C₃-perfluoroalkyl, chloro, C₁-C₆-alkylthio, C₁-C₆-alkoxy, nitro, cyano, and cyano-C₁-C₃-alkyl; or

wherein CR^{b}R^{c} forms a 3-6 membered ring;

wherein R¹ is selected from C₁-C₃-perfluoroalkyl, chloro, C₁-C₆-alkylthio, C₁-C₆-alkoxy, nitro, cyano, and cyano-C₁-C₃-alkyl;

wherein R² is selected from carboxyl, aminocarbonyl, C₁-C₆-alkylsulfonylaminocarbonyl, and C₁-C₆-alkoxycarbonyl;

wherein R³ is selected from hydrido, phenyl, thienyl, C₁-C₆-alkyl, and C₂-C₆-alkenyl;

wherein R⁴ is one or more radicals independently selected from hydrido, halo, C₁-C₆-alkyl, C₂-C₆-alkenyl, C₂-C₆-alkynyl, halo-C₂-C₆-alkylnyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkoxy, heteroaryl-C₁-C₆-alkoxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl)-C₁-C₃-hydroxyalkyl, C₁-C₆-hydroxyalkyl, hydroxyimino-C₁-C₆-alkyl, C₁-C₆-alkylamino, arylamino, N-aryl-N—C₁-C₆-alkylamino, heteroarylamino, N-heteroaryl-N—C₁-C₆-alkylamino, nitro, cyano, amino, aminosulfonyl, C₁-C₆-alkylaminosulfonyl, arylaminosulfonyl, heteroarylaminosulfonyl, N-aryl-C₁-C₆-alkylaminosulfonyl, N-heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl, and C₁-C₆-alkylcarbonyl; or wherein R⁴ together with the ring to which it is attached forms a radical selected from naphthyl, quinolyl, isoquinolyl, quinolizinyl, quinoxalinyl, and dibenzofuryl; and

wherein the A ring atoms A¹, A², A³, and A⁴ are independently selected from carbon and nitrogen with the proviso that at least two of A¹, A², A³, and A⁴ are carbon.

Some chromene compounds useful as COX-2 selective inhibitors in the present invention are shown in Table 3, including the diastereomers, enantiomers, racemates, tautomers, salts, esters, amides and prodrugs thereof.

TABLE 3
Examples of Chromene COX-2 Selective Inhibitors as Embodiments

| COM- | |
|---|---|
| POUND | |
| NUMBER | STRUCTURAL FORMULA |
| A-6 | 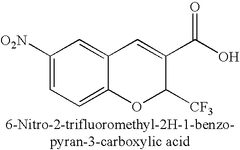 |
| A-7 | 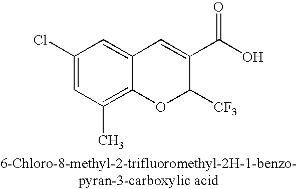 |
| A-8 | 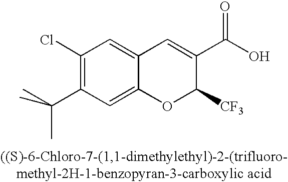 |
| A-9 | 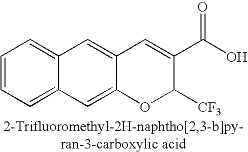 |
| A-10 | 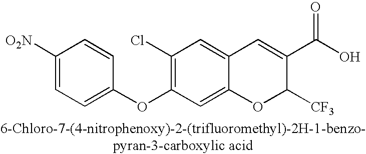 |
| A-11 | 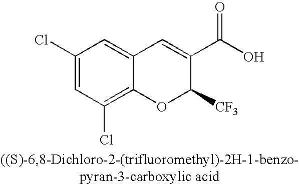 |
| A-12 | 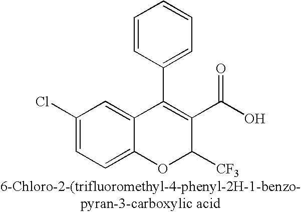 |
| A-13 | 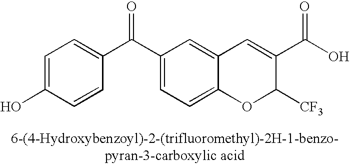 |
| A-14 | 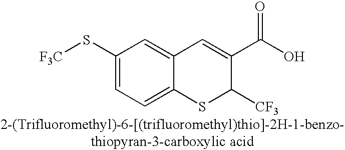 |
| A-15 | 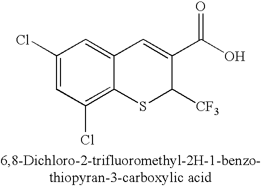 |
| A-16 | 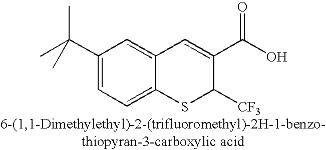 |
| A-17 | 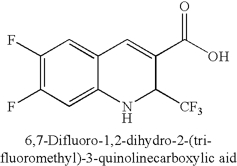 |
| A-18 | 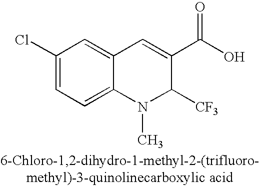 |
| A-19 | 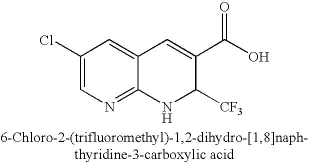 |
| A-20 | 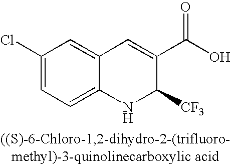 |

The individual patent documents referenced in Table 4 below describe the preparation of the COX-2 inhibitors of Table 3.

TABLE 4
References for Preparation of Chromene COX-2 Inhibitors

| COMPOUND | |
|---|---|
| NUMBER | PATENT REFERENCE |
| A-6 | U.S. Pat. No. 6,077,850; example 37 |
| A-7 | U.S. Pat. No. 6,077,850; example 38 |
| A-8 | U.S. Pat. No. 6,077,850; example 68 |
| A-9 | U.S. Pat. No. 6,034,256; example 64 |
| A-10 | U.S. Pat. No. 6,077,850; example 203 |
| A-11 | U.S. Pat. No. 6,034,256; example 175 |
| A-12 | U.S. Pat. No. 6,077,850; example 143 |
| A-13 | U.S. Pat. No. 6,077,850; example 98 |
| A-14 | U.S. Pat. No. 6,077,850; example 155 |
| A-15 | U.S. Pat. No. 6,077,850; example 156 |
| A-16 | U.S. Pat. No. 6,077,850; example 147 |
| A-17 | U.S. Pat. No. 6,077,850; example 159 |
| A-18 | U.S. Pat. No. 6,034,256; example 165 |
| A-19 | U.S. Pat. No. 6,077,850; example 174 |
| A-20 | U.S. Pat. No. 6,034,256; example 172 |

In a further preferred embodiment of the invention, the cyclooxygenase inhibitor is selected from the class of tricyclic cyclooxygenase-2 selective inhibitors represented by the general structure of Formula:

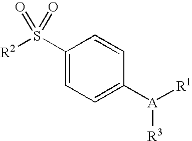

wherein A is a substituent selected from partially unsaturated or unsaturated heterocyclyl and partially unsaturated or unsaturated carbocyclic rings;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl or amino; and

wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt or derivative or prodrug thereof.

In a still more preferred embodiment of the invention, the cyclooxygenase-2 selective inhibitor represented by the above formula is selected from the group of compounds, illustrated in Table 5, consisting of celecoxib (A-21), valdecoxib (A-22), deracoxib (A-23), rofecoxib (A-24), etoricoxib (MK-663; A-25), JTE-522 (A-26), or a pharmaceutically acceptable salt or derivative or prodrug thereof.

In an even more preferred embodiment of the invention, the COX-2 selective inhibitor is selected from the group consisting of celecoxib, rofecoxib and etoricoxib.

TABLE 5
Examples of Tricyclic COX-2 Selective Inhibitors as Embodiments

| COMPOUND | |
|---|---|
| NUMBER | STRUCTURAL FORMULA |
| A-21 | 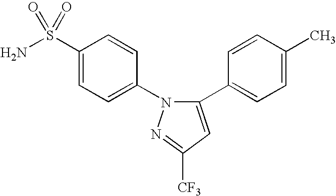 |
| A-22 | 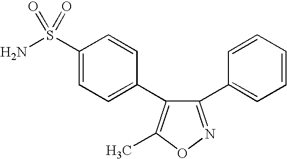 |
| A-23 | 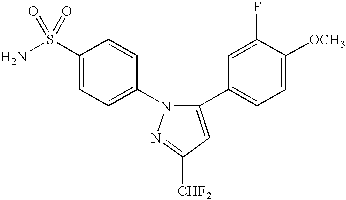 |
| A-24 | 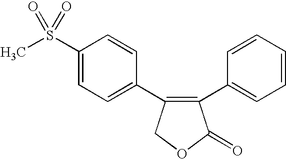 |
| A-25 | 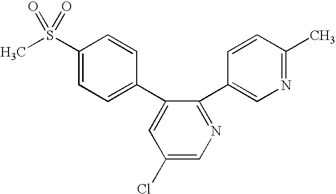 |
| A-26 | 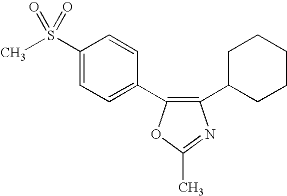 |
| A-27 | 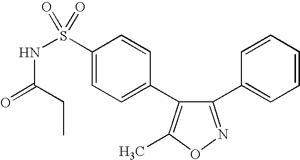 |

The individual patent documents referenced in Table 6 below describe the preparation of the aforementioned cyclooxygenase-2 selective inhibitors A-21 through A-27.

TABLE 6
Documents for Preparation of Tricyclic COX-2 Inhibitors and
Prodrugs

| COMPOUND | |
|---|---|
| NUMBER | PATENT REFERENCE |
| A-21 | U.S. Pat. No. 5,466,823 |
| A-22 | U.S. Pat. No. 5,633,272 |
| A-23 | U.S. Pat. No. 5,521,207 |
| A-24 | U.S. Pat. No. 5,840,924 |
| A-25 | PCT Publ. No. WO 98/03484 |
| A-26 | PCT Publ. No. WO 00/25779 |
| A-27 | U.S. Pat. No. 5,932,598 |

U.S. Pat. No. 6,180,651 describes COX-2 selective inhibitors of the diarylmethylidene furan derivative that are useful in the combination of the present invention. In a preferred embodiment of the present invention, the diarylmethylidene furan derivative COX-2 selective inhibitor is BMS-347070.

Routes of Administration

In therapeutic use for treating, or combating, bacterial infections in a mammal (e.g., animals, humans), an antibiotic or a pharmaceutically acceptable salt thereof, and a cyclooxygenase inhibitor or a pharmaceutically acceptable salt or derivative or prodrug thereof can each be administered orally, parenterally, topically, rectally, or intranasally.

Parenteral administrations include injections to generate a systemic effect or injections directly to the afflicted area. Examples of parenteral administrations are subcutaneous, intravenous, intramuscular, intradermal, intrathecal, intraocular, intravetricular, and general infusion techniques.

Topical administrations include the treatment of infectious areas or organs readily accessibly by local application, such as, for example, eyes, ears including external and middle ear infections, vaginal, open and sutured or closed wounds, and skin. Topical administrations also include transdermal delivery to generate a systemic effect.

Rectal administrations include, for example, the form of suppositories.

Intranasal administrations include, for example, nasal aerosol and inhalation applications.

Preferred routes of administration include, for example, oral and intravenous administration.

Pharmaceutical compositions of an antibiotic or a pharmaceutically acceptable salt thereof, and a cyclooxygenase inhibitor or a pharmaceutically acceptable salt or derivative or prodrug thereof, may be prepared by methods well known in the art, including, for example, conventional mixing, dissolving, granulation, dragee-making, levigating, emulsifying, encapsulating, entrapping, lyophilizing processes, and spray drying.

Pharmaceutical compositions for use in accordance with the present invention may be formulated in a conventional manner using one or more physiologically acceptable carriers including, for example, excipients and auxiliaries that facilitate processing of the active compounds into preparations that can be used pharmaceutically. Proper formulation is dependent upon the route of administration chosen.

For oral administration, compounds can be formulated by combining active compounds with pharmaceutically acceptable carriers well known in the art. Such carriers enable compounds disclosed in the present application to be formulated as tablets, pills, lozenges, dragees, capsules, liquids, solutions, emulsions, gels, syrups, slurries, suspensions and the like, for oral ingestion by a patient. A carrier can be at least one substance that may also function, for example, as a diluent, flavoring agent, solubilizer, lubricant, suspending agent, binder, tablet disintegrating agent, or encapsulating agent. Examples of such carriers or excipients include, for example, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols, and other pharmaceutical acceptable materials.

Dragee cores are preferably provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for uses including, for example, identification and characterization of different combinations of active compound doses.

Pharmaceutical compositions that can be used orally include, for example, push-fit capsules made of gelatin, as well as soft, sealed capsules made of gelatin and a plasticizer (e.g., glycerol and sorbitol). The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may also be added in these formulations.

Liquid form compositions include, for example, solutions, suspensions, and emulsions. For example, there may be provided solutions of compounds disclosed in the present application dissolved in water and water-propylene glycol and water-polyethylene glycol systems, optionally containing suitable conventional coloring agents, flavoring agents, stabilizers, and thickening agents.

Compounds may also be formulated for parenteral administration, including, for example, injections, bolus injections, and continuous infusion. Formulations for parenteral administration may be presented in unit dosage form including, for example, ampoules and multi-dose containers, optionally with an added preservative. The compositions may take such forms as suspensions, solutions or emulsions in oily or aqueous vehicles, and may contain formulating materials such as suspending, stabilizing, and/or dispersing agents.

For injection, compounds disclosed in the present application are preferably formulated in aqueous solution, preferably in physiologically compatible buffers or physiological saline buffer. Suitable buffering agents include, for example, trisodium orthophosphate, sodium bicarbonate, sodium citrate, N-methylglucamine, L(+)-lysine and L(+)-arginine.

The compounds or compositions can also be administered intravenously or intraperitoneally by, for example, infusion or injection. Solutions of the active compound or its salts can be prepared in water, optionally mixed with a nontoxic surfactant. Dispersions can also be prepared in glycerol, liquid polyethylene glycols, triacetin, and mixtures thereof, and in oils. Under ordinary conditions of storage and use, these preparations may contain a preservative to prevent the growth of microorganisms.

Pharmaceutical dosage forms suitable for injection or infusion include, for example, sterile aqueous solutions or dispersions, or sterile powders including the active ingredient that are adapted for the extemporaneous preparation of sterile injectable or infusible solutions or dispersions, optionally encapsulated in liposomes. In all cases, the ultimate dosage form is preferably sterile, fluid, and stable under the conditions of manufacture and storage. The liquid carrier or vehicle is preferably a solvent or liquid dispersion medium including, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. Prevention of the action of microorganisms can be brought about by various antibacterial and antifungal agents including, for example, parabens, chlorobutanol, phenol, sorbic acid, thimerosal, and the like. In many cases, it will be preferable to include isotonic agents including, for example, sugars, buffers, or sodium chloride. Prolonged absorption of the injectable compositions can be brought about by the use of agents to delay absorption (e.g., aluminum monostearate, gelatin) in the compositions.

Sterile injectable solutions can be prepared by incorporating an active compound in the required amount in the appropriate solvent with optional ingredients as required (e.g., as enumerated above), followed by, for example, filter sterilization. In the case of sterile powders for the preparation of sterile injectable solutions, preferred methods of preparation include, for example, vacuum drying and freeze drying techniques, which yield a powder of the active ingredient plus any additional desired ingredient present in the previously sterile- filtered solutions.

Other parenteral administrations also include aqueous solutions of a water-soluble form, such as, without limitation, a salt, of the active compound. Additionally, suspensions of the active compounds may be prepared in a lipophilic vehicle. Suitable lipophilic vehicles include, for example, fatty oils such as sesame oil, synthetic fatty acid esters such as ethyl oleate and triglycerides, and materials such as liposomes. Aqueous injection suspensions preferably contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, an active ingredient may be in a powder form for constitution with a suitable vehicle (e.g., sterile, pyrogen-free water) before use.

For suppository administration, compounds may also be formulated by mixing the agent with a suitable non-irritating excipient that is solid at room temperature but liquid at rectal temperature, which will therefore melt in the rectum to release the drug. Such materials include, for example, cocoa butter, beeswax, and other glycerides.

For administration by inhalation, compounds disclosed in the present application are preferably conveniently delivered through an aerosol spray in the form of solution, dry powder, or cream. The aerosol may use, for example, a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, a pharmaceutical composition may be formulated in a suitable ointment containing the active component suspended or dissolved in one or more carriers. Carriers for topical administration of the compounds disclosed in the present application include, for example, mineral oil, liquid petrolatum, white petrolatum, propylene glycol, polyoxyethylene, polyoxypropylene compound, emulsifying wax, and water. Aternatively, the pharmaceutical compositions can be formulated in suitable lotions, including, for example, suspensions, emulsions, and creams containing the active components suspended or dissolved in one or more pharmaceutically acceptable carriers. Suitable carriers include, for example, mineral oil, sorbitan monostearate, polysorbate 60, cetyl esters wax, ceteary alcohol, 2-octyldodecanol, benzyl alcohol, and water.

For ophthalmic and otitis uses, pharmaceutical compositions may be formulated as micronized suspensions in isotonic, pH adjusted sterile saline, or preferably, as solutions in isotonic, pH adjusted sterile saline, either with or without a preservative such as benzylalkonium chloride. Alternatively, for ophthalmic uses, pharmaceutical compositions may be formulated in an ointment such as petrolatum.

In addition to the formulations described previously, the compounds may also be formulated as depot preparations. Such long acting formulations may be in the form of implants. Compounds disclosed in the present application may be formulated for this route of administration with suitable polymers, hydrophobic materials, or as a sparing soluble derivative such as, without limitation, a sparingly soluble salt.

Additionally, compounds may be delivered using a sustained-release system. Various sustained-release materials have been established and are well known by those skilled in the art. Sustained-release capsules may, depending on their chemical nature, preferably release the compounds for up to about 24 hours, and more preferably for up to several days. Depending on the chemical nature and the biological stability of the therapeutic reagent, additional strategies for protein stabilization may be employed.

An antibiotic or a pharmaceutically acceptable salt thereof, and a cyclooxygenase inhibitor or a pharmaceutically acceptable salt or derivative or prodrug thereof may each be administrated intravenously in the form of an aqueous solution. Preferred antibiotics for this IV aqueous solution include, for example, linezolid, amikacin, gentamicin, tobramycin, imipenem, meropenem, cefotetan, cefoxitin, cefuroxime, cefoperazone, cefotaxime, ceftazidime, ceftozoxime, ceftriaxone, cefepime, azithromycin, ampicillin, mezlocillin, piperacillin, ticarcillin, ciprofloxacin, levofloxacin, alatrofloxacin, gatifloxacin, minocycline, chloramphenicol, clindamycin, vancomycin, cefazolin, penicillin G, nafcillin, ofloxacin, and oxacillin.

An aqueous solution for IV administration can be placed in the container that is selected from the group consisting of a bag, a bottle, a vial, a large volume parenteral, a small volume parenteral, a prefilled syringe, and a cassette. It is realized that a vial is a bottle. However, those skilled in the art use the term “bottle” to refer to larger bottles and “vials” to refer to smaller bottles. It is preferred that the container be a bag, a bottle, a vial, or a prefilled syringe. It is more preferred that the container be a bag or bottle. It is most preferred that the container be a bag. The shape and/or size of the container are unimportant. It is preferred that the container be a bag sufficient to hold 25 to 2,000 mL of IV solution. It is preferred that the compounds be put in bags in amounts of 100, 200, or 300 mL of solution. However, smaller or larger volumes are acceptable.

It is well known to those skilled in the art that an IV solution must be sterile. While there are a number of methods to sterilize an IV solution, it is preferred to terminally moist heat or steam sterilize IV solutions including compounds disclosed in the present application. When the term terminally “moist heat sterilize” is used, it refers to and includes steam sterilization.

When terminally moist heat sterilizing an IV solution, the solution is preferably placed in the container in which (1) it will be stored and then transferred to the container from which it will ultimately be administered, or (2) stored and then ultimately administered from the same container to deliver the IV solution to the patient. Therefore, it is preferable that compounds disclosed in the present application do not react with the container in which they are to be terminally moist heat sterilized and stored/stored-administered.

The preferred dosage and frequency of administration of an aqueous pharmaceutical composition depends on the particular combinations of compounds being used, the particular condition being treated, the severity of the condition being treated, the age, weight, general physical condition of the particular patient, and other medication the individual may be taking, as is well known to those skilled in the art. The preferred dosage and frequency of administration can be more accurately determined by measuring the blood level or concentration of the compounds in the patient's blood and/or the patient's response to the particular condition being treated.

Without further elaboration, it is believed that one skilled in the art can, using the preceding description, practice the present invention to its fullest extent. The present invention is illustrated by the following examples. It is to be understood that the particular examples, materials, amounts, and procedures are to be interpreted broadly in accordance with the scope and spirit of the invention as set forth herein. Those skilled in the art will promptly recognize appropriate variations from the procedures both as to reactants and as to reaction conditions and techniques.

### EXAMPLES

### Example 1

A pharmaceutically effective amount of linezolid and a pharmaceutically effective amount of celecoxib is administered to a mammal to treat or prevent a bacterial infection. The combination therapy results in reduced side effects resulting from the administration of the antibiotic.

### Example 2

A pharmaceutically effective amount of linezolid and a pharmaceutically effective amount of rofecoxib is administered to a mammal to treat or prevent a bacterial infection. The combination therapy results in reduced side effects resulting from the administration of the antibiotic.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating or preventing a bacterial infection in a mammal comprising administering to said mammal in need
(a) a pharmaceutically effective amount of an antibiotic or a pharmaceutically acceptable salt thereof; and
(b) a pharmaceutically effective amount of a cyclooxygenase inhibitor or a pharmaceutically acceptable salt or derivative or prodrug thereof.

2. The method of claim 1 wherein the infection is caused by gram-positive bacteria.

3. The method of claim 1 wherein the infection is caused by gram-negative bacteria.

4. The method of claim 1 wherein the antibiotic is linezolid, amikacin, gentamicin, spectinomycin, tobramycin, imipenem/cilastatin combination, meropenem, cefadroxil, cefazolin, cephalexin, cefaclor, cefotetan, cefoxitin, cefprozil, cefuroxime, loracarbef, cefdinir, cefixime, cefoperazone, cefotaxime, cefpodoxime, ceftazidime, ceftibuten, ceftozoxime, ceftriaxone, cefepime, azithromycin, clarithromycin, dirithromycin, penicillin G, cloxacillin, dicloxacillin, nafcillin, oxacillin, amoxicillin, amoxicillin/clavulanic acid combination, ampicillin, ampicillin/sulbactam combination, mezlocillin, piperacillin, piperacillin/tazobactam combination, ticarcillin, ticarcillin/clavulanate combination, nalidixic acid, ciprofloxacin, enoxacin, lomefloxacin, norfloxacin, ofloxacin, levofloxacin, sparfloxacin, alatrofloxacin, gatifloxacin, moxifloxacin, trimethoprim/sulfamethoxazole combination, sulfisoxazole, sulfamethoxazole, doxycycline, minocycline, tetracycline, chloramphenicol, clindamycin, quinupristin/dalfopristin combination, fosfomycin, nitrofurantoin, rifampin, trimethoprim, vancomycin, or combinations thereof.

5. The method of claim 1 wherein the antibiotic is amikacin, gentamicin, spectinomycin, tobramycin, imipenem/cilastatin combination, meropenem, cefaclor, cefotetan, cefoxitin, cefprozil, cefuroxime, loracarbef, cefdinir, cefixime, cefoperazone, cefotaxime, cefpodoxime, ceftazidime, ceftibuten, ceftozoxime, ceftriaxone, cefepime, azithromycin, clarithromycin, dirithromycin, amoxicillin, amoxicillin/clavulanic acid combination, ampicillin, ampicillin/sulbactam combination, mezlocillin, piperacillin, piperacillin/tazobactam combination, ticarcillin, ticarcillin/clavulanate combination, nalidixic acid, ciprofloxacin, enoxacin, lomefloxacin, norfloxacin, ofloxacin, levofloxacin, sparfloxacin, alatrofloxacin, gatifloxacin, moxifloxacin, trimethoprim/sulfamethoxazole combination, sulfisoxazole, sulfamethoxazole, doxycycline, minocycline, tetracycline, chloramphenicol, aztreonam, fosfomycin, nitrofurantoin, trimethoprim, or combinations thereof.

6. The method of claim 1 wherein the antibiotic is linezolid.

7. The method of claim 1 wherein the cyclooxygenase inhibitor is a cyclooxygenase-2 selective inhibitor.

8. The method of claim 7 wherein the cyclooxygenase-2 selective inhibitor is meloxicam, RS-57067, ABT-963, COX-189, NS-398, BMS-347070, and combinations thereof.

9. The method of claim 7 wherein the cyclooxygenase-2 selective inhibitor is represented by the general formula 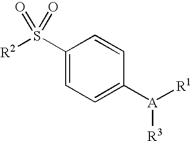
wherein
A is a partially unsaturated heterocyclic ring, unsaturated heterocyclic rings, partially unsaturated carbocyclic ring, or unsaturated carbocyclic ring;
R¹ is at least one substituent selected from heterocyclyl, cycloalkyl, cycloalkenyl and aryl; wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is methyl or amino; and
R³ is hydride, halide, alkyl, alkenyl, alkynyl, oxo, cyanide, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

10. The method of claim 7 wherein the cyclooxygenase-2 selective inhibitor is celecoxib, valdecoxib, deracoxib, rofecoxib, etoricoxib, JTE-522, or combinations thereof.

11. The method of claim 7 wherein the cyclooxygenase-2 selective inhibitor is celecoxib, rofecoxib, or combination thereof.

12. The method of claim 7 wherein the cyclooxygenase-2 selective inhibitor is celecoxib.

13. The method of claim 7 wherein the cyclooxygenase-2 selective inhibitor is refecoxib.

14. The method of claim 1 wherein the antibiotic is linezolid and the cyclooxygenase inhibitor is celecoxib, rofecoxib, or combination thereof.

15. The method of claim 1 wherein the antibiotic is linezolid and the cyclooxygenase inhibitor is celecoxib.

16. The method of claim 1 wherein the antibiotic is linezolid and the cyclooxygenase inhibitor is rofecoxib.

17. The method of claim 7 wherein the cyclooxygenase-2 selective inhibitor is a chromene structural class compound has the general formula: 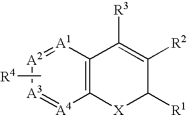
wherein
X is O, S, CR^{c}R^{b} or NR^{a};
R^{a} is hydrido, C₁-C₃-alkyl, phenyl-C₁-C₃-alkyl, (substituted phenyl)-C₁-C₃-alkyl, C₁-C₃-alkoxycarbonyl-C₁-C₃-alkyl, or carboxy-C₁-C₆-alkyl;
R^{b} and R^{c} is independently hydrido, C₁-C₃-alkyl, substituted or unsubstituted phenyl-C₁-C₃-alkyl, C₁-C₃-perfluoroalkyl, chloro, C₁-C₆-alkylthio, C₁-C₆-alkoxy, nitro, cyano, or cyano-C₁-C₃-alkyl; or wherein CR^{b}R^{c} forms a 3-6 membered ring;
R¹ is C₁-C₃-perfluoroalkyl, chloro, C₁-C₆-alkylthio, C₁-C₆-alkoxy, nitro, cyano,
or
cyano-C₁-C₃-alkyl;
R² is carboxyl, aminocarbonyl, C₁-C₆-alkylsulfonylaminocarbonyl, and C₁-C₆-alkoxycarbonyl;
R³ is hydrido, phenyl, thienyl, C₁-C₆-alkyl, and C₂-C₆-alkenyl;
wherein R⁴ is one or more radicals independently selected from hydrido, halo, C₁-C₆-alkyl, C₂-C₆-alkenyl, C₂-C₆-alkynyl, halo-C₂-C₆-alkylnyl, aryl-C₁-C₃-alkyl,
aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkoxy, heteroaryl-C₁-C₆-alkoxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl)-C₁-C₃-hydroxyalkyl, C₁-C₆-hydroxyalkyl, hydroxyimino-C₁-C₆-alkyl, C₁-C₆-alkylamino, arylamino,
N-aryl-N—C₁-C₆-alkylamino, heteroarylamino, N-heteroaryl-N—C₁-C₆-alkylamino, nitro, cyano, amino, aminosulfonyl, C₁-C₆-alkylaminosulfonyl, arylaminosulfonyl, heteroarylaminosulfonyl, N-aryl-C₁-C₆-alkylaminosulfonyl, N-heteroaryl-C₁-C₆-alkylaminosulfonyl, heterocyclylsulfonyl, C₁-C₆-alkylsulfonyl,
aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl, or C₁-C₆-alkylcarbonyl; or wherein R⁴ together with the ring to which it is attached form from naphthyl, quinolyl, isoquinolyl, quinolizinyl, quinoxalinyl, and dibenzofuryl; and
the A ring atoms A¹, A², A³, and A⁴ are independently selected from carbon and nitrogen with the proviso that at least two of A¹, A², A³, and A⁴ are carbon.

18. The method of claim 17 wherein the chromene structural class compound is a substituted benzopyran, a substituted benzothiopyran, a substituted dihydroquinoline, or a substituted dihydronaphthyridine.

19. The method of claim 18 wherein the substituted benzopyran is 6-nitro-2-trifluoromethyl-2H-1-benzopyran-3-carboxylic acid;
6-chloro-8-methyl-2-trifluoromethyl-2H-1-benzopyran-3-carboxylic acid;
(S)-6-chloro-7-(1,1-dimethylethyl)-2-trifluoromethyl-2H-1-benzopyran-3-carboxylic acid; 2-trifluoromethyl-2H-naphtho[2,3-b]pyran-3-carboxylic acid;
6-chloro-7-(4-nitrophenoxy)-2-trifluoromethyl-2H-1-benzopyran-3-carboxylic acid;
(S)-6,8-dichloro-2-trifluoromethyl-2H-1-benzopyran-3-carboxylic acid;
6-chloro-2-trofluoromethyl-4-phenyl-2H-1-benzopyran-3-carboxylic acid;
6-(4-hydroxybenzoyl)-2-trifluoromethyl-2H-1-benzopyran-3-carboxylic acid; or combinations thereof.

20. The method of claim 18 wherein the substituted benzothiopyran is 2-trifluoromethyl-6-[(trifluoromethyl)thiol]-2H-1-benzothiopyran-3-carboxylic acid;
6,8-dichloro-2-trifluoromethyl-2H-1-benzothiopyran-3-carboxylic acid;
6-(1,1-dimethylethyl)-2-trifluoromethyl-2H-1-benzothiopyran-3-carboxylic acid; or combinations thereof.

21. The method of claim 18 wherein the substituted dihyroquinoline is
6,7-difluoro-1,2-dihydro-2-trifluoromethyl-3-quinolinecarboxylic acid;
6-chloro-1,2-dihydro-1-methyl-2-trifluoromethyl-3-quinolinecarboxylic acid;
(S)-6-chloro-1,2-dihydro-2-trifluoromethyl-3-quinolinecarboxylic acid; or combinations thereof.

22. The method of claim 18 wherein the substituted dihydronaphthyridine is
6-chloro-2-trifluoromethyl-1,2-dihydro[1,8]naphthyridine-3-carboxylic acid.

23. The method of claim 1 wherein the mammal is a human.

24. The method of claim 1 wherein the antibiotic or pharmaceutically acceptable salt thereof is administered orally, parenterally, topically, rectally, or intranasally.

25. The method of claim 24 wherein the antibiotic is linezolid.

26. The method of claim 1 wherein the cyclooxygenase inhibitor or pharmaceutically acceptable salt or derivative or prodrug thereof is administered orally, parenterally, topically, rectally, or intranasally.

27. The method of claim 26 wherein the cyclooxygenase inhibitor is a cyclooxygenase-2 selective inhibitor.

28. The method of claim 27 wherein the cyclooxygenase-2 selective inhibitor is celecoxib.

29. The method of claim 1 wherein (a) the antibiotic or pharmaceutically acceptable salt thereof and (b) the cyclooxygenase inhibitor or pharmaceutically acceptable salt or derivative or prodrug thereof are administered concurrently.

30. The method of claim 29 wherein the antibiotic is linezolid; and the cylooxygenase inhibitor is a cyclooxygenase-2 selective inhibitor.

31. The method of claim 30 wherein the cyclooxygenase-2 selective inhibitor is celecoxib.

32. The method of claim 1 wherein (a) the antibiotic or pharmaceutically acceptable salt thereof and (b) the cyclooxygenase inhibitor or pharmaceutically acceptable salt or derivative or prodrug thereof are administered concomitantly.

33. The method of claim 32 wherein the antibiotic is linezolid; and the cylooxygenase inhibitor is a cyclooxygenase-2 selective inhibitor.

34. The method of claim 33 wherein the cyclooxygenase-2 selective inhibitor is celecoxib.

35. The method of claim 1 wherein (a) the antibiotic or pharmaceutically acceptable salt thereof and (b) the cyclooxygenase inhibitor or pharmaceutically acceptable salt or derivative or prodrug thereof are administered at least once per day.

36. The method of claim 35 wherein the antibiotic is linezolid; and the cylooxygenase inhibitor is a cyclooxygenase-2 selective inhibitor.

37. The method of claim 36 wherein the cyclooxygenase-2 selective inhibitor is celecoxib.

38. A method for reducing side effects of an antibiotic in a mammal comprising:
(a) administering to the mammal a sufficient amount of an antibiotic or a pharmaceutically acceptable salt thereof; and
(b) administering to the mammal a pharmaceutically effective amount of a cyclooxygenase-selective inhibitor or a pharmaceutically acceptable salt or derivative or prodrug thereof.

39. The method of claim 38 wherein the antibiotic is linezolid and the cyclooxygenase inhibitor is a cyclooxygenase-2 selective inhibitor.

40. The method of claim 39 wherein the cyclooxygenase-2 selective inhibitor is celecoxib, rofecoxib, or combination thereof.

41. The method of claim 39 wherein the cyclooxygenase-2 selective inhibitor is celecoxib.

42. A composition comprising:
an antibiotic or a pharmaceutically acceptable salt thereof; and
an effective amount of a cyclooxygenase-2 selective inhibitor or a pharmaceutically acceptable salt or derivative or prodrug thereof.

43. The composition of claim 42 wherein the antibiotic is linezolid and the cyclooxygenase-2 selective inhibitor is celecoxib, rofecoxib, or combination thereof.

44. The method of claim 42 wherein the cyclooxygenase-2 selective inhibitor is celecoxib.

45. A medical kit comprising:
a container;
an antibiotic or a pharmaceutically acceptable salt thereof in the container; and an effective amount of a cyclooxygenase-2 selective inhibitor or a pharmaceutically acceptable salt or derivative or prodrug thereof in the container.

46. The medical kit of claim 45 wherein the antibiotic is linezolid and the cyclooxygenase-2 selective inhibitor is celecoxib, rofecoxib, or combination thereof.

47. The medical kit of claim 45 wherein the cyclooxygenase-2 selective inhibitor is celecoxib.
